(19) United States
(12) Patent Application Publication
(10) Pub. No.: US 2010/0098670 A1
(43) Pub. Date: Apr. 22, 2010

(54) Autologous dental pulp stem cell-based bone graft substitute

(75) Inventor: Xuebin Yang, Leeds (GB)

Correspondence Address: CLARK & ELBING LLP, 101 FEDERAL STREET, BOSTON, MA, 02110, US

(73) Assignee: Cerapedics Inc., Westminster, CO (US)

(21) Appl. No.: 12/313,522

(22) Filed: Nov. 21, 2008

Related U.S. Application Data
(60) Provisional application No. 61/004,014, filed on Nov. 21, 2007.

(51) Int. Cl. A61K 35/12; C12N 5/00; A61P 19/00

(52) U.S. Cl. 424/93.7; 435/377; 435/375; 435/395

(57) ABSTRACT

The invention features methods and compositions for promoting the growth and differentiation of dental pulp stem cells and the use of the differentiated cells for the treatment of orthopedic conditions.

### CROSS REFERENCE TO RELATED APPLICATIONS

This application claims benefit from U.S. provisional application, Ser. No. 61/004,014, filed Nov. 21, 2007, and incorporated herein by reference.

### BACKGROUND OF THE INVENTION

Bone has a remarkable capacity for growth, regeneration, and remodeling. This capacity is largely due to the induction of osteoblasts that are recruited to sites of new bone formation. The process of recruitment remains unclear, although the immediate environment of the cells is likely to play a role via cell-matrix-osteoinductive factor—cell interactions (see Reddi, A. H. Tissue Eng. 6:351 (2000); Rezania et al., J. Orthop. Res. 17:615 (1999); Rose et al., Biochem. Biophys. Res. Commun. 292:1 (2002); Langer, R., and Vacanti, J. P. Science 260:920 (1993); Lutolf et al., Nat. Biotechnol. 21:513 (2003); Zandonella, C. Nature 421:884 (2003); Dayoub et al., Tissue Eng. 9:347 (2003); Cancedda et al., Matrix Biol. 22:81 (2003); and Baylink et al., J. Bone Miner. Res. 8(Suppl. 2):S565 (1993)). The central and first step to successful tissue engineering is the ability of cells to adhere to an extracellular material followed by the ability of the cells to differentiate, leading to the production and organization of an extracellular matrix. Tremendous effort has centered on the improvement of cell adhesion with a variety of materials. However, the immediate limitation for many polymer materials is the absence of a chemically reactive pendent chain for the easy attachment of cells, drugs, cross-linkers, or biologically active moieties (Yang et al., Tissue Eng. 7:679 (2001)). Generally, cell adhesion is a series of interactive events comprising (1) initial cell attachment, (2) cell spreading, (3) organization of an actin cytoskeleton, and (4) formation of focal adhesions (LeBaron et al., Tissue Eng. 6:85 (2000)). The attachment of the cell to the extracellular matrix is known to be exquisitely controlled by various families of adhesion receptors, including the integrins, selectins, cadherins, and immunoglobulins (Ruoslahti et al., Science 238:491 (1987); Hutmacher et al., Int. J. Periodontics Restorative Dent. 21:49 (2001); Stock et al., Annu. Rev. Med. 52:443 (2001); and Muschler et al., Clin. Orthop. 395:66 (2002)).

The generation of biomimetic microenvironments, using scaffolds containing cell recognition sequences in combination with bone forming cells, offers tremendous potential for skeletal tissue regeneration. Although a number of different methods have been developed to meet such a clinical requirement, to date most common procedures still rely on bone grafts (Goldberg et al., Semin. Arthroplasty 4:58 (1993)). Fresh autogenous and allogeneic bone grafts, both cancellous and cortical, provide a source of osteoprogenitor cells, osteoinductive growth factors, and a structural scaffold for new bone formation. Furthermore, the three-dimensional framework of both autografts and allografts can function as mechanical supports for angiogenesis and the invasion of osteoprogenitor cells into the bone grafts. However, the use of autograft material is limited by the loss of structure in donor and fresh allografts can induce both local and systemic immune responses that diminish or destroy the osteoinductive and conductive processes (see Goldberg et al., Semin. Arthroplasty 4:58 (1993); Betz, R. R. Orthopedics 25:s561 (2002); and Yang et al., Bone 29:523 (2001).

During tooth formation, interactions between epithelial and dental papilla cells promote tooth morphogenesis by stimulating a subpopulation of mesenchymal cells to differentiate into odontoblasts, which in turn form primary dentin. Morphologically, odontoblasts are columnar polarized cells with eccentric nuclei and long cellular processes aligned at the outer edges of dentin (Smith et al., Int. J. Dev. Biol. 39:273 (1995)). After tooth eruption, reparative dentin is formed by odontoblasts in response to general mechanical erosion or disruption, and through dentinal degradation caused by bacteria (Kitamura et al., J. Dent. Res. 78:673 (1999)). These odontoblasts arise from the proliferation and differentiation of a precursor population of human dental pulp stem cell (HDPSCs) residing within the pulp tissue. These isolated postnatal human DPSCs have been shown to form a dentin-pulp-like complex upon implantation (Gronthos et al., Proc Natl Acad Sci USA 97:13625 (2000)).

The development of an ideal bone graft substitute for skeletal tissue repair/regeneration is a major clinical need. The goal of this study was to investigate the effect of P-15 on human dental pulp stem cell (HDPSCs) growth and differentiation leading to bone formation and to develop autologous mesenchymal stem cell based bone graft substitute for skeletal tissue repair/regeneration.

There is an urgent need for the development of bone graft substitutes incorporating easily harvested osteogenic autologous cells.

### SUMMARY OF THE INVENTION

Applicants have discovered that human dental pulp stem cells (HDPSCs) can be differentiated along an osteogenic lineage in the presence of P-15 peptide. The resulting cultures provide an autologous osteogenic source of cells which can be used in a bone graft substitute.

Accordingly, in a first aspect the invention features a method for promoting the differentiation of dental pulp stem cells into osteogenic cells in vitro by contacting the dental pulp stem cells with a biocompatible calcified substrate having a collagen mimetic deposited thereon in an amount sufficient to promote the differentiation.

In a related aspect the invention features a method for increasing the growth of dental pulp stem cells in vitro by contacting the dental pulp stem cells with a biocompatible calcified substrate having a collagen mimetic deposited thereon in an amount sufficient to increase the growth.

The invention further features a method for treating an orthopedic condition in a subject by (i) providing a dental pulp stem cell; (ii) culturing the dental pulp stem cell in vitro in the presence of a biocompatible calcified substrate having a collagen mimetic deposited thereon to produce differentiated osteogenic cells; and (iii) implanting the osteogenic cells into the subject in an amount effective to treat the orthopedic condition.

The invention also features a method for correcting bone deficiencies at a bone repair site in a subject by (i) providing dental pulp stem cells; (ii) culturing the dental pulp stem cells in vitro in the presence of a biocompatible calcified substrate having a collagen mimetic deposited thereon to produce differentiated osteogenic cells; and (iii) implanting the osteogenic cells into the subject in an amount effective to treat the bone repair site. The method can be used, for example, in maxillofacial surgery, facial reconstructive surgery, or for correcting periodontal defects.

In certain embodiments of the above methods, the dental pulp stem cell is an autologous cell. In other embodiments, the dental pulp stem cell is an allogenous cell. The dental pulp stem cell may be an isolated cell, or part of a cell population which contains dental pulp stem cells, such as dental pulp stromal cells.

The invention features a kit including (i) a biocompatible calcified substrate having a collagen mimetic deposited thereon, and (ii) instructions for contacting dental pulp stem cells in vitro with the substrate to produce osteogenic cells.

The invention also features a kit including (i) a biocompatible calcified substrate having a collagen mimetic deposited thereon, and (ii) instructions for contacting dental pulp stem cells in vitro with the substrate to increase the growth of the dental pulp stem cells.

In certain embodiments, the kits of the invention can further include instructions for implanting the calcified substrate into a subject. In other embodiments, the kits of the invention can further include instructions for implanting the calcified substrate into a subject having an orthopedic condition. The orthopedic condition to be treated can be any orthopedic condition described herein. In certain embodiments, the kits of the invention can further include instructions for use in maxillofacial surgery, facial reconstructive surgery, or filling periodontal defects.

In still another aspect, the invention features a container, such as a flask, dish, tube, beaker, or plate, for growing cells in vitro including (i) a biocompatible calcified substrate having a collagen mimetic deposited thereon and (ii) dental pulp stem cells.

In an embodiment of any of the above aspects of the invention, the calcified substrate is selected from mineralized bone matrix, deorganified bone matrix, anorganic bone matrix, or a mixture thereof. Desirably, the calcified substrate is anorganic bone matrix. In another embodiment of any of the above aspects of the invention, the collagen mimetic is a peptide described herein. Desirably, the collagen mimetic is P-15.

By “BMSC” is meant a bone marrow mesenchyme-derived stem cell. BMSCs are also referred to as “bone marrow stem cells” and “bone marrow multipotent progenitor cells.”

By “dental pulp stem cell” or “DPSC” is meant a stem cell which exhibit a similar expression pattern as BMSCs for a variety of markers (i.e., CD14−, C34−, CD44+, CD45−, MyoD-, neurofilament-, collagen-II-, PPARγ-, integrin β1+, VCAM-1+, among others), but distinguished from BMSCs in that bone sialoprotein (a bone matrix protein) is absent in DPSC cultures, but present at low levels in BMSC cultures. Furthermore, DPSCs are not adipogenic or are very weakly adipogenic. The characterization and isolation of DPSCs is described in U.S. Pat. No. 7,052,907, incorporated herein by reference.

By “stem cell” is meant a cell capable of (i) self renewing, and (ii) producing multiple differentiated cell types.

“Administering,” “introducing,” “implanting,” and “transplanting” are used interchangeably and refer to the placement of the differentiated DPSCs or bone graft substitutes of the invention into a subject, e.g., a human subject, by a method or route which results in localization of the cells at a desired site.

By “collagen mimetic” is meant a synthetic peptide having a domain that includes—Ile-Ala—folded in a beta-bend at physiologic conditions and that mimics cell binding by collagen and has enhanced cell binding in comparison to collagen. The collagen mimetics which can be used in the compositions and methods of the invention includes all or part of the peptide of SEQ ID NO. 1 (also known as “P-15”), includes all or part of 15 amino acid residues, Gly-Thr-Pro-Gly-Pro-Gln-Gly-Ile-Ala-Gly-Gln-Arg-Gly-Val-Val (SEQ ID NO. 1) of the α1(I) chain of collagen, and spans approximately residues 766-780 of this chain. Collagen mimetics of the invention include Gly-Thr-Pro-Gly-Pro-Gln-Gly-Ile-Ala-Gly-Gln-Arg-Gly-Val-Val (SEQ ID NO: 1), Gly-Pro-Gln-Gly-Ile-Ala-Gly-Gln-Arg (SEQ ID NO: 2), Gln-Gly-Ile-Ala-Gly-Gln (SEQ ID NO: 3), Gln-Gly-Ile-Ala-Gly-Gln-Arg (SEQ ID NO: 4), Phe-Gly-Ile-Ala-Gly-Phe (SEQ ID NO: 5), Gly-Ile-Ala-Gly-Gln (SEQ ID NO: 6), Gln-Gly-Ala-Ile-Ala-Gln (SEQ ID NO: 7), Phe-Gly-Ile-Ala-Gly-Phe (SEQ ID NO:8), Cys-Gly-Ile-Ala-Gly-Cys (SEQ ID NO:9), (SEQ ID NO:10), N-Acetyl Ile-Ala-Ala (SEQ ID NO:11), Ile-Ala-.beta.Ala (SEQ ID NO:12), and N-Acetyl Ile-Ala NMe (SEQ ID NO:13), and any other collagen mimetics described in U.S. Pat. No. 7,199,103, incorporated herein by reference.

As used herein, the terms “an amount sufficient” and “sufficient amount” refer to the amount of collagen mimetic required to either differentiate DPSCs along an osteogenic lineage (i.e., produce osteogenic cells from DPSCs) or the amount of collagen mimetic required to increase the growth of DPSCs in comparison to the same conditions but in the absence of collagen mimetic.

As used herein, the terms “an amount effective” refers to the amount of osteogenic cells produced using the methods of the invention required to treat or prevent an orthopedic condition or for correcting a bone deficiency at a bone repair site in a subject. An orthopedic condition or bone deficiency is treated where a subject experiences, for example, an increase in ossification, reduced healing time for the repair of a bone defect, and/or the strengthening of existing bone (i.e., to reduce the future incidents of fracture). The effective amount of osteogenic cells used to practice the invention for therapeutic or prophylactic treatment of orthopedic conditions varies depending upon the manner of administration, the age, body weight, and general health of the subject. Ultimately, the attending physician will decide the appropriate amount and proper route of administration or implantation. Such amount is referred to as an “effective” amount.

DPSCs for use in the methods, kits, and compositions of the invention can be obtained from a variety of sources and can be classified according to the genetic relationship between the DPSC source and the subject being treated. As used herein, the term “allogenous DPSCs” refers to DPSCs obtained from same species, but a different genotype than that of the subject receiving treatment. The term “autologous DPSCs” refers to DPSCs obtained from same species and having the same genotype as that of the subject receiving treatment.

Other features and advantages of the invention will be apparent from the following detailed description, the drawings, and the claims.

### BRIEF DESCRIPTION OF THE DRAWINGS

FIG. 1 is a fluorescent microscope image of HDPSCs outgrowth from dental pulp explants cultured with ABM-P-15 (A) and ABM alone (B). The arrow shows viable cells on the scaffold. Magnification ×100. Enhanced numbers of cells grown out from the pulp tissue explants and cell bridge formation between adjacent scaffold particles in the ABM-P-15 group compared to ABM alone (FIG. 1B).

FIG. 2 is a confocal microscope image of HDPSCs outgrown from the pulp explants onto the ABM-P-15 (A) and ABM (B) scaffolds (magnification ×200). P-15 enhanced the outgrowth of HDPSCS from the pulp explants and promoted cell bridge formation between scaffold particles (FIG. 2A). In contrast, HDPSCs on the ABM only (control) scaffolds were around the individual scaffold particles only (FIG. 2B).

FIG. 3 is an SEM image of dental pulp cultured with ABM-P-15 (A) or ABM alone (B). HDPSCs had outgrown from pulp explants onto the ABM-P-15 particles and had formed clusters and cell bridges to link the scaffold particles (FIG. 3A). In comparison, only a few cells appeared on the surface of individual ABM only scaffold particles (FIG. 3B).

FIG. 4 is photograph depicting alkaline phosphatase (ALP) of dental pulp explants cultured with ABM-P-15 (A) and ABM (B). The long arrows show the pulp explants themselves, which were stained positively (red) for ALP in the presence or absence of P-15. The short arrow head shows ALP positive staining of HDPSCs on ABM-P-15 particles adjacent to the explants. Magnification ×40. The cells outgrown from the explants on to the scaffold particles themselves were stained positively for ALP in the ABM-P-15 group. In contrast, cells on the ABM alone scaffold particles did not show evidence of ALP activity, which was restricted to the explants themselves.

FIG. 5 is a photograph depicting confocal microscopy of HDPSC growth in the presence of and absence of P-15. Live/dead fluorescent markers showed HDPSCs growth on ABM-P-15 (A) and ABM (B) scaffolds. Enhanced HDPSCs attachment and spreading on ABM-P-15 scaffolds were observed compared to ABM alone.

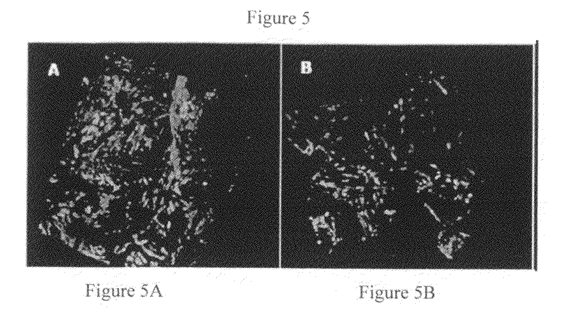

FIG. 6 is a photograph depicting alkaline phosphatase (ALP) and toluidine blue staining of hDPSC cultures. Toluidine blue counter staining on ALP stained scaffolds showed that majority of cells on ABM-P-15 were ALP positive, while majority of the cells on ABM were ALP negative. The enhanced expression of alkaline phosphatase (ALP) showed that P-15 promoted the HDPSCs differentiation along the osteogenic lineage.

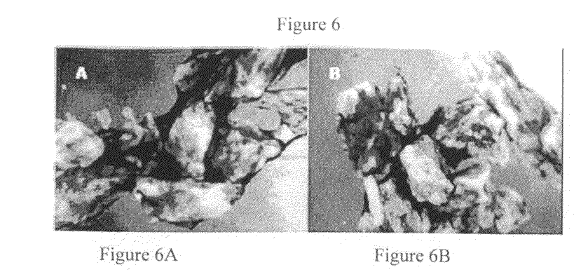

### DETAILED DESCRIPTION

The invention provides methods and compositions for promoting the growth and differentiation of dental pulp stem cells and the use of the differentiated cells for the treatment of orthopedic conditions.

### Calcified Substrates

The methods and compositions of the invention include a calcified substrate having a collagen mimetic deposited thereon. The calcified substrate can be, for example, selected from calcium phosphate materials, such as mineralized bone matrix, deorganified bone matrix, anorganic bone matrix, or a mixture thereof.

The calcium phosphate may be any biocompatible, calcium phosphate material known in the art. The calcium phosphate material may be produced by any one of a variety of methods and using any suitable starting components. For example, the calcium phosphate material may include amorphous, apatitic calcium phosphate. Calcium phosphate material may be produced by solid-state acid-base reaction of crystalline calcium phosphate reactants to form crystalline hydroxyapatite solids. Other methods of making calcium phosphate materials are known in the art, some of which are described below.

### Crystalline Hydroxyapatite

Alternatively, the calcium phosphate material can be crystalline hydroxyapatite (HA). Crystalline HA is described, for example, in U.S. Pat. Nos. Re. 33,221 and Re. 33,161. These patents teach preparation of calcium phosphate remineralization compositions and of a finely crystalline, non-ceramic, gradually resorbable hydroxyapatite carrier material based on the same calcium phosphate composition. A similar calcium phosphate system, which consists of tetracalcium phosphate (TTCP) and monocalcium phosphate (MCP) or its monohydrate form (MCPM), is described in U.S. Pat. Nos. 5,053,212 and 5,129,905. This calcium phosphate material is produced by solid-state acid-base reaction of crystalline calcium phosphate reactants to form crystalline hydroxyapatite solids.

Carbonate substituted crystalline HA materials (commonly referred to as dahllite) may be prepared (see U.S. Pat. No. 5,962,028). These HA materials (commonly referred to as carbonated hydroxyapatite) can be formed by combining the reactants with an aqueous liquid to provide a substantially uniform mixture, shaping the mixture as appropriate, and allowing the mixture to harden in the presence of water. During hardening, the mixture crystallizes into a solid and essentially monolithic apatitic structure.

The reactants will generally include a phosphate source, e.g., phosphoric acid or phosphate salts, an alkali earth metal, particularly calcium, optionally crystalline nuclei, particularly hydroxyapatite or calcium phosphate crystals, calcium carbonate, and a physiologically acceptable lubricant. The dry ingredients may be pre-prepared as a mixture and subsequently combined with aqueous liquid ingredients under conditions where substantially uniform mixing occurs.

### P-15 Coated Anorganic Bone Mineral Matrix

The P-15 coated ABM particles have a mean particle diameter of 300 microns, and nearly all will fall within a range between 200 microns to 425 microns. However, a particle size range between 50 microns to 2000 microns may also be used.

Anorganic bone mineral matrix (ABM) may also be a synthetic alloplast matrix or some other type of xenograft or allograft mineralized matrix that might not fit the definition of “anorganic.” The alloplast could be a calcium phosphate material or it could be one of several other inorganic materials that have been used previously in bone graft substitute formulations, e.g., calcium carbonates, calcium sulphates, calcium silicates, or mixtures thereof that could function as biocompatible, osteoconductive matrices. The anorganic bone mineral matrix, synthetic alloplast matrix, and xenograft or allograft mineralized matrix are collectively referred to as the osteoconductive component.

### Implantation

The compositions of the invention can be used in the preparation of bone graft substitutes which are implanted into a subject. Because the compositions of the invention are mixed with tissues which are rich in stem cells and/or bone forming cells or seeded with bone forming cells, such as stem cells and/or osteoprogenitor cells, and/or osteoblasts, the compositions promote ossification.

The compositions of the invention can be useful for repairing a variety of orthopedic conditions. For example, the compositions may be injected into the vertebral body for prevention or treatment of spinal fractures, injected into long bone or flat bone fractures to augment the fracture repair or to stabilize the fractured fragments, or injected into intact osteoporotic bones to improve bone strength. The compositions can be useful in the augmentation of a bone-screw or bone-implant interface. Additionally, the compositions can be useful as bone filler in areas of the skeleton where bone may be deficient. Examples of situations where such deficiencies may exist include post-trauma with segmental bone loss, post-bone tumor surgery where bone has been excised, and after total joint arthroplasty (e.g., impaction grafting and so on). The compositions may be formulated as a paste prior to implantation to hold and fix artificial joint components in patients undergoing joint arthroplasty, as a strut to stabilize the anterior column of the spine after excision surgery, as a structural support for segmented bone (e.g., to assemble bone segments and support screws, external plates, and related internal fixation hardware), and as a bone graft substitute in spinal fusions.

The compositions of the invention can be used to coat prosthetic bone implants. For example, where the prosthetic bone implant has a porous surface, the composition may be applied to the surface to promote bone growth therein (i.e., bone ingrowth). The composition may also be applied to a prosthetic bone implant to enhance fixation within the bone.

The compositions of the invention can be used as a remodelling implant or prosthetic bone replacement, for example in orthopoedic surgery, including hip revisions, replacement of bone loss, e.g. in traumatology, remodelling in maxillofacial surgery or filling periodontal defects and tooth extraction sockets, including ridge augmentation. The compositions of the invention may thus be used for correcting any number of bone deficiencies at a bone repair site.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Methods

The HDPSCs were isolated from human dental pulp tissues and cultured on ABM and ABM-P-15 scaffolds in media. The cultured cells on the scaffolds were analysed by live/dead fluorescent markers, histological staining (ALP, Alizalin Red, Toluidine blue), confocal microscopy and scanning electron microscopy. The effect of P-15 on HDPSCs BMP-2 production was confirmed by a well developed promyoblast induction assay via co-culturing of C2C12 promyoblasts and HDPSCs on ABM or ABM-P-15. The effect of ABM-P-15 on cell outgrowth and attachment behaviour, of human dental pulp explants was demonstrated by mixing human dental pulp with ABM and/or ABM-P-15 scaffolds and culturing in vitro. Cell outgrowth from the explants was assessed by live/dead fluorescent markers, histological staining (ALP), confocal microscope and scanning electron microscopy.

Human Dental Pulp Tissue Preparation

Teeth were obtained with patients' informed consent following project approval by the NHS local ethical committee (COREC: 06/Q1206/165). Human dental pulp was extracted from sound intact teeth, which had been surgically removed at the Leeds Dental Institute for clinical reasons. Each tooth was washed within a Class II hood and cracked in a bench vice. The dental pulp tissues were harvested and washed with 1×PBS and minced into small pieces (1×2×2 mm³) which were kept in the PBS and ready for use.

In Vitro Model

PepGen P-15 (ABM-P15) and/or Osteo-Graf/N-300 (ABM alone) were provided by Cerapedics Inc. (Lakewood, Colo.) in a particulate form. 50 mg of ABM-P-15 and ABM alone were transferred into the 48 well plates and sterilized under UV radiation for 30 minutes. Minced human dental pulp explants were mixed with the scaffolds and cultured in basal media at 37° C. within a 5% CO₂ incubator. The culture medium was changed every 5 days. Samples were fixed at various time points for further analysis including live (Cell-tracker green)/dead fluorescent marker (at 2 weeks and 6 weeks), confocal microscopy, SEM and alkaline phosphatase (ALP) staining.

### Results

Enhanced HDPSCs attachment and spreading on ABM-P-15 scaffolds were observed compared to ABM alone. Live/dead fluorescent images showed that extensive cell bridges formed between the ABM-P-15 particles resulting in aggregation of the scaffold particles. The enhanced expression of alkaline phosphatase (ALP) showed that P-15 had promoted HDPSCs differentiation along the osteogenic lineage. Toluidine blue counter staining on ALP stained scaffolds showed that the majority of cells on ABM-P-15 were ALP positive, while the majority of the cells on ABM were ALP negative. Sandersons rapid bone staining showed that ABM-P-15 promoted new bone matrix formation compare to ABM alone. Increased BMP-2 production was confirmed based on the observation of enhanced ALP expression by C2C12 cells co-cultured in the presence of HDPSCs on ABM-P-15 particles compared to ABM alone. RT-PCR showed that ABM-P-15 enhanced HDPSCs expression for type 1 collagen, alkaline phosphatase, RUNX2, and osteocalcin by HDPSCs. Culture of human dental pulp explants showed that ABM-P-15 enhanced both outgrowth of HDPSCs from the explants and cell bridge formation across adjacent scaffolds particles compared with ABM alone.

The Effect of P-15 on the Outgrowth of HDPSCs from the Explants

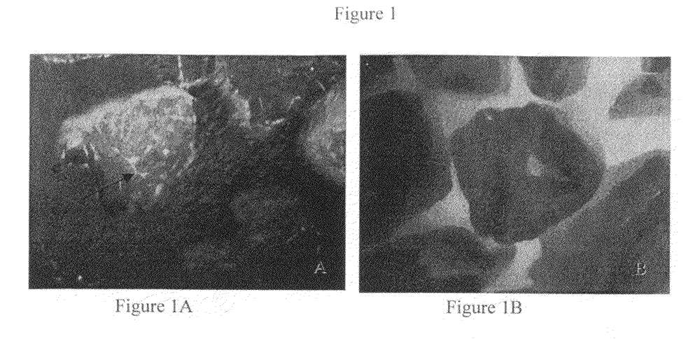

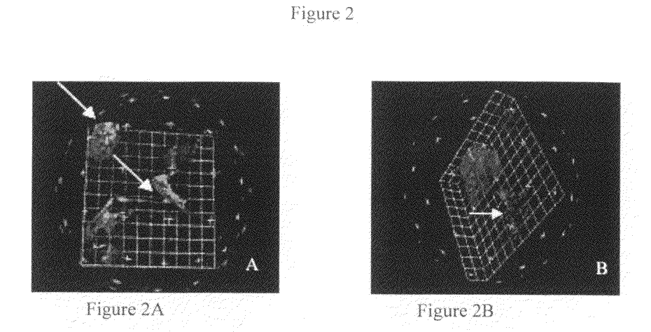

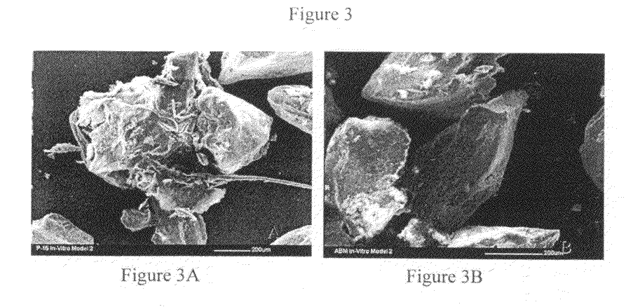

Fluorescent live/dead markers were used to visualize the cell outgrowth from human dental pulp tissues in the presence or absence of P-15. FIG. 1A shows enhanced numbers of cells grown out from the pulp tissue explants and cell bridge formation between adjacent scaffold particles in the ABM-P-15 group compared to ABM alone (FIG. 1B). Confocal microscopic images confirmed that P-15 enhanced the outgrowth of HDPSCS from the pulp explants and promoted cell bridge formation between scaffold particles (FIG. 2A). In contrast, HDPSCs on the ABM only (control) scaffolds were around the individual scaffold particles only (FIG. 2B). Scanning electron microscopic images confirmed that HDPSCs had outgrown from pulp explants onto the ABM-P-15 particles and had formed clusters and cell bridges to link the scaffold particles (FIG. 3A). In comparison, only a few cells appeared on the surface of individual ABM only scaffold particles (FIG. 3B).

ALP Staining of Human Dental Pulp Outgrown from Dental Pulp Tissues

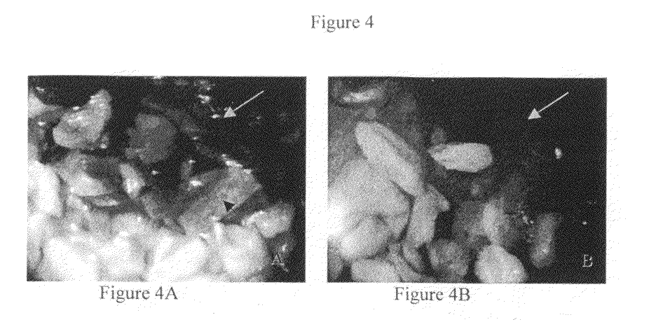

The osteogenic induction potential of ABM-P-15 was investigated by determination of the expression of ALP. FIG. 4 shows that the dental pulp explants themselves stained for positive ALP in both ABM-P-15 and ABM only groups. However, the cells outgrown from the explants on to the scaffold particles themselves were stained positively for ALP in the ABM-P-15 group. Any cells on the ABM alone scaffold particles did not show evidence of ALP activity, which was restricted to the explants themselves FIG. 4B).

### Conclusion

These results show that P-15 adsorbed ABM particles provide an ideal biomimetic microenvironment for HDPSCs chemotaxis, growth and differentiation along the osteogenic lineage, thus making this scaffold a good candidate material scaffold for bone regeneration.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general; the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for promoting the differentiation of dental pulp stem cells into osteogenic cells in vitro, said method comprising contacting said dental pulp stem cells with a biocompatible calcified substrate having a collagen mimetic deposited thereon in an amount sufficient to promote said differentiation.

2. A method for increasing the growth of dental pulp stem cells in vitro, said method comprising contacting said dental pulp stem cells with a biocompatible calcified substrate having a collagen mimetic deposited thereon in an amount sufficient to increase said growth.

3. A method for treating an orthopedic condition in a subject, said method comprising:
(i) providing a dental pulp stem cell;
(ii) culturing said dental pulp stem cell in vitro in the presence of a biocompatible calcified substrate having a collagen mimetic deposited thereon to produce differentiated osteogenic cells; and
(iii) implanting said osteogenic cells into said subject in an amount effective to treat said orthopedic condition.

4. A method for correcting bone deficiencies at a bone repair site said method comprising:
(i) providing dental pulp stem cells;
(ii) culturing said dental pulp stem cells in vitro in the presence of a biocompatible calcified substrate having a collagen mimetic deposited thereon to produce differentiated osteogenic cells; and
(iii) implanting said osteogenic cells into said subject in an amount effective to treat said bone repair site.

5. The method of claim 4, for use in maxillofacial surgery, facial reconstructive surgery, or correcting periodontal defects.

6. The method of claim 3 or 4, wherein said dental pulp stem cell is an autologous cell or an allogenous cell.

7. The method of claim any of claims 1-4, wherein said calcified substrate is selected from mineralized bone matrix, deorganified bone matrix, anorganic bone matrix, or a mixture thereof.

8. The method of any of claims 1-4, wherein said collagen mimetic is P-15.

9. A kit comprising (i) a biocompatible calcified substrate having a collagen mimetic deposited thereon, and (ii) instructions for contacting dental pulp stem cells in vitro with said substrate to produce osteogenic cells.

10. A kit comprising (i) a biocompatible calcified substrate having a collagen mimetic deposited thereon, and (ii) instructions for contacting dental pulp stem cells in vitro with said substrate to increase the growth of said dental pulp stem cells.

11. The kit of claim 8 or 9, further comprising instructions for implanting said calcified substrate into a subject.

12. The kit of claim 10, further comprising instructions for implanting said calcified substrate into a subject having an orthopedic condition.

13. The kit of claim 10, further comprising instructions for use in maxillofacial surgery, facial reconstructive surgery, or correcting periodontal defects.

14. The kit of claim 8 or 9, wherein said calcified substrate is anorganic bone matrix and said collagen mimetic is P-15.

15. A container for growing cells in vitro comprising (i) a biocompatible calcified substrate having a collagen mimetic deposited thereon and (ii) dental pulp stem cells, wherein said calcified substrate is anorganic bone matrix and said collagen mimetic is P-15.


SEQUENCE LISTING (13 sequences)

SEQ ID NO: 1 - protein, 15 aa, Artificial Sequence
  Synthetic Construct
GTPGPQGIAGQRGVV

SEQ ID NO: 2 - protein, 9 aa, Artificial Sequence
  Synthetic Construct
GPQGIAGQR

SEQ ID NO: 3 - protein, 6 aa, Artificial Sequence
  Synthetic Construct
QGIAGQ

SEQ ID NO: 4 - protein, 7 aa, Artificial Sequence
  Synthetic Construct
QGIAGQR

SEQ ID NO: 5 - protein, 6 aa, Artificial Sequence
  Synthetic Construct
FGIAGF

SEQ ID NO: 6 - protein, 5 aa, Artificial Sequence
  Synthetic Construct
GIAGQ

SEQ ID NO: 7 - protein, 6 aa, Artificial Sequence
  Synthetic Construct
QGAIAQ

SEQ ID NO: 8 - protein, 6 aa, Artificial Sequence
  Synthetic Construct
FGIAGF

SEQ ID NO: 9 - protein, 6 aa, Artificial Sequence
  Synthetic Construct
CGIAGC

SEQ ID NO: 10 - protein, 6 aa, Artificial Sequence
  Synthetic Construct
EGIAGK

SEQ ID NO: 11 - protein, 3 aa, Artificial Sequence
  Synthetic peptide
XAA

SEQ ID NO: 12 - protein, 3 aa, Artificial Sequence
  Synthetic peptide
IAX

SEQ ID NO: 13 - protein, 2 aa, Artificial Sequence
  Synthetic Peptide
XX